(19) United States
(12) Patent Application Publication
(10) Pub. No.: US 2019/0209284 A1
(43) Pub. Date: Jul. 11, 2019

(54) ORIENTATION MARKERS FOR FLUOROSCOPIC VISUALIZATION

(71) Applicant: Cook Medical Technologies LLC, Bloomington, IN (US)

(72) Inventors: Blayne A. Roeder, Bloomington, IN (US); Mark Svendsen, Bloomington, IN (US)

(73) Assignee: Cook Medical Technologies LLC, Bloomington, IN (US)

(21) Appl. No.: 15/867,332

(22) Filed: Jan. 10, 2018

(51) Int. Cl. A61F 2/07; A61B 90/00; A61F 2/856; A61F 2/966

(52) U.S. Cl. CPC A61F 2/07; A61B 90/39; A61F 2002/9505; A61F 2/966; A61F 2/856

(57) ABSTRACT

A stent graft device having a plurality of axially offset rows of radiopaque markers for rotational orientation of the stent graft device. The stent graft device may include an expandable stent frame and attached graft member. Each row of radiopaque markers may include a plurality of radiopaque markers that are evenly spaced. Each row may also include a length that covers a limited portion of the circumference of the stent graft device, where each row is circumferentially offset such that a fluoroscopic projection of the stent graft device illustrates one of a plurality of unique visual orientations of the axially offset rows for use in visually determining a current rotational orientation of the stent graft device.

### BACKGROUND

The present disclosure relates generally to apparatuses and methods for treating vascular conditions, and more specifically, to apparatuses and methods for aiding alignment of a medical device in a vessel.

An aortic aneurysm is a disease condition in which the aorta (the large artery coming off the left side of the heart) is abnormally dilated. Because aortic aneurysms can rupture and be fatal, either surgical or endovascular approaches may be required for treatment. Endovascular approaches are less invasive and thus often preferred over surgical approaches. Endovascular approaches usually involve the placement of a covered stent graft in a preferred orientation inside the aneurysm to maintain blood flow through the aorta while diverting blood away from the aneurysm.

An X-ray is usually the mode of endovascular visualization and there can be challenges with seeing and orienting a stent graft or other medical implement with an X-ray device during an endovascular procedure.

### BRIEF SUMMARY

In order to address the challenges of visualizing and orienting a stent graft or other medical implement during an endovascular procedure, a system and method for providing improved visualization and orientation under an imaging system such as an X-ray is provided.

According to one aspect, a stent graft device is provided that includes a stent frame having a central axis. A generally tubular graft member is attached to the stent frame. The stent graft device has a compressed state and an expanded state, wherein a diameter of the stent graft device in the expanded state is greater than that of the stent graft device in the compressed state. The stent graft device includes a first row of radiopaque makers positioned along only a first portion of a circumference of the stent graft device and a second row of radiopaque markers positioned along only a second portion of the circumference of the stent graft device. Each of the radiopaque markers of the first and second rows may be positioned on one of the stent frame or the graft member. The first row is axially and circumferentially offset from the second row such that a unique rotational position of the stent graft device is detectable via a pattern formed by the first and second rows of radiopaque markers in an image of the stent graft device.

According to another aspect, A stent graft device includes a plurality of stent frame elements arranged along a central axis. A graft member may be attached with the stent frame elements, where the stent graft device has a compressed state and an expanded state, and where a diameter of the stent graft in the expanded state is greater than that of the stent graft device in the compressed state. The stent graft device may further include a plurality of rows of radiopaque markers positioned along a circumference of the stent graft device. Each of the plurality of rows of radiopaque markers is axially spaced apart from each other of the plurality of rows. Each of the radiopaque markers is positioned on one of the plurality of stent frame elements or the graft member. Also, each of the plurality of rows has a circumferential length less than a total circumference of the stent graft device, where a unique rotational position of the stent graft device is detectable via a pattern formed by the plurality of rows of radiopaque markers in an image of the stent graft device.

In yet another aspect, a stent graft device is disclosed that includes a radially expandable stent frame having a central axis. The stent graft device also may include a tubular graft member attached with the radially expandable stent frame, where the stent graft device has a compressed state and an expanded state, and where a diameter of the stent graft device in the expanded state is greater than that of the stent graft device in the compressed state. The stent graft device may include a plurality of rows of radiopaque markers positioned on the stent graft device at axially offset locations. Each of the plurality of rows may consist of at least one radiopaque marker positioned along a circumferentially unique portion of a total circumference of the stent graft device such that a fluoroscopic projection of the stent graft device on a two-dimensional display visually indicates a rotational orientation of the stent graft device in increments of 90 degrees or less. The at least one radiopaque marker for each row may be a single continuous radiopaque marker extending along the respective circumferentially unique portion of the total circumference occupied by the particular row, or it may be a plurality of equally spaced radiopaque markers.

### BRIEF DESCRIPTION OF THE DRAWINGS

FIG. 1A is a perspective view of one embodiment of an uncompressed stent graft with alignment markers arranged as described herein.

FIG. 1B is a perspective view of the stent graft of FIG. 1B in a compressed state.

FIG. 2A is a side view of an x-ray projection of the stent graft of FIG. 1B in a desired rotational orientation.

FIG. 2B is the x-ray projection of the stent graft of FIG. 1B when it is rotated 90° from the desired rotational orientation of FIG. 2A.

FIG. 2C is the x-ray projection of the stent graft of FIG. 1B when it is rotated 180° from the desired rotational orientation of FIG. 2A.

FIG. 2D is the x-ray projection of the stent graft of FIG. 1B when it is rotated 270° from the desired rotational orientation of FIG. 2A.

### DETAILED DESCRIPTION

A stent graft device is disclosed herein that permits improved visualization of the position, including rotational orientation, of the stent graft during insertion and placement in a body.

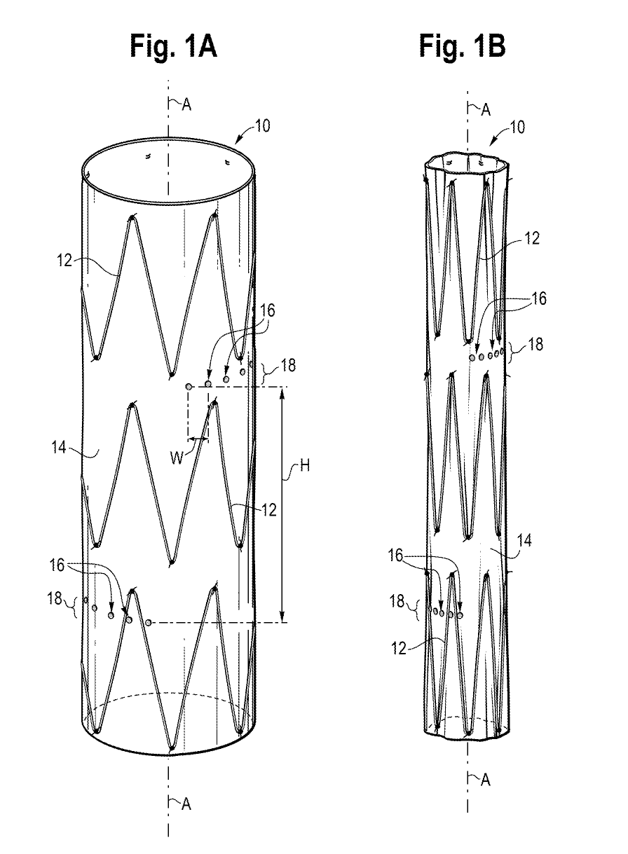

Referring now to FIGS. 1A and 1B, an embodiment of stent graft 10 having a radiopaque alignment system for use in aligning and positioning the stent graft in a vessel is illustrated. The stent graft 10 is shown in an expanded, also referred to as deployed, position in FIG. 1A, while the same stent graft 10 is illustrated in a compressed, also referred to as undeployed, position in FIG. 1B. The stent graft 10 may include one or more sets of expandable stent frame elements 12 aligned along a central axis A that together define a stent frame. The inner portion of each stent frame element 12 facing the interior of the stent graft device 10 may also be referred to herein as the luminal surface and the exterior portion of each stent frame element 12 may also be referred to herein as the abluminal surface of the stent frame element 12. The stent frame elements 12 may be positioned inside or on the tubular graft portion 14, utilizing any of a number of known stent wire and graft materials, and attached together to form the stent graft 10.

The radiopaque alignment system for the stent graft 10 may include at least two axially offset and circumferentially uniquely positioned rows 18 of radiopaque markers 16. The series of radiopaque markers 16 within each row 18 may be placed along the stent graft 10 in a circumferential pattern having a noticeable distance W between each of the markers 16 when in the expanded position (FIG. 1A), as well as an axial spacing H. When the stent graft 10 is in the contracted, or undeployed position (see FIG. 1B), the markers 16 in a given row 18 are circumferentially spaced closer to one another due to the smaller overall circumference of the stent graft 10. In one implementation, the markers 16 in a given row 18 are all equally circumferentially spaced apart. In other implementations, the markers 16 in a given row 18 may be spaced at differing distances apart from one another over the partial circumferential length of the stent graft that the row is covering.

Within a given row 18 of markers 16, the markers can be placed in a straight horizontal line (as shown in FIGS. 1A and 1B) or in a curved/non-straight line pattern. It should be noted that the straight horizontal orientation refers to a direction along the circumference that is perpendicular to the axis A of the stent graft 10. Thus the straight line of a row 18 follows the curve of the circumference of the stent graft as shown in FIGS. 1A and 1B, but would appear straight when viewed from outside of the stent graft in an x-ray image taken at an angle perpendicular to the axis A of the stent graft 10. Any of a number of materials that are readily visible under an imaging scan, such as an x-ray scan (i.e., radiopaque materials), for example metals such as gold, may be used for the radiopaque markers 16. The markers 16 may be attached to the stent graft 10 (to either or both of the stent frame elements 12 or tubular graft portion 14) through welding, winding, weaving, printing, adhering or any of a number of known mechanisms for attaching materials to a stent graft.

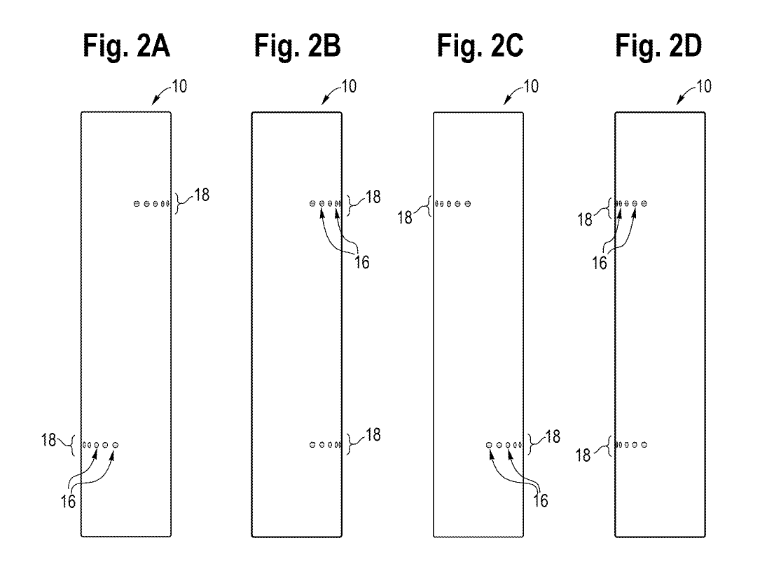

FIGS. 2A-2D shows a sample x-ray projections of the compressed (undeployed) stent graft 10 of FIG. 1B in different rotational orientations about the central axis of the stent graft 10. More specifically, FIGS. 2A-2D illustrate an alignment technique using partial circumferential rows 18 of radiopaque markers 16 that are axially and circumferentially offset from one another. In the example of FIGS. 2A-2D, each row 18 only has 25% circumferential coverage of the stent graft 10 (i.e., in a given row 18 from the first to the last marker 16 there is coverage of 90° out of the 360° stent graft circumference). When looking at the deployed, uncompressed image of the graft of FIG. 1A, it is noted that the two rows do not follow the same circumferential path (i.e., marker row #1 goes counterclockwise around the stent graft 10 and marker row #2 goes clockwise around the stent graft at a different axial position along the stent graft). In other words, the rows are not at the same axial position so that they take different paths that can be visually differentiated by an observer via a fluoroscope.

Assuming the uncompressed stent of FIG. 1A is shown in the “correct” orientation in which the stent graft should be positioned during deployment, then the stent graft 10 in the compressed state should look like the image shown in FIG. 2A. If the stent graft 10 is rotated any amount, as in the sequence of 90° clockwise rotational orientations from the initial orientation of FIG. 2A through the final orientation of FIG. 2D, a unique projection of the radiopaque markers 16 is seen. FIG. 2B shows the stent graft 10 rotated 90° from FIG. 2A, FIG. 2C illustrates a projection of the stent graft at a 180° rotation from the orientation of FIG. 2A, and the projection of FIG. 2D represents a projection of the stent graft when rotated 270° clockwise (or 90° counterclockwise) from the original orientation of FIG. 2A. Each of the four orientations of FIGS. 2A-2D would be seen as distinct orientations when viewed via x-ray and would thus provide feedback to the user that adjustments may be needed for proper positioning.

Assuming the orientation of FIG. 2A is the desired orientation, any other orientation of the rows 18 of markers 16 would indicate misalignment and the amount of misalignment. It should be noted that in other embodiments, the “correct” orientation could be the projection of FIGS. 2B, 2C, or 2D, or some another projection with all the other projections as incorrect projections. When inserting the stent graft 10 into a vessel in a body, a medical professional may look for one of the predefined, unique arrangements markers, such as shown in FIGS. 2A-2D, as the stent graft compressed stent graft is being maneuvered into position in the vessel. When the desired visibly identifiable arrangement of the rows 18 of markers is attained, then the stent graft may be expanded and fixed in place in the vessel.

Another utility of the design described in this example is that it does not overlap with any stents and can thus be more easily packaged in the compressed (undeployed) state. For example the radiopaque markers 16 in each row 18 may be individually sewn onto the material of the tubular graft portion 14 and not attached to the stent frame elements 12 so that folding of the stent graft 10 is not affected. Alternatively, the radiopaque markers 16 may be attached directly to the stent frame elements 12 where the stent graft 10 may not fold as neatly. In different implementations, the radiopaque material may be a metal or non-metal radiopaque material. The radiopaque material may be in the form of a thread or suture (e.g. polypropylene that is impregnated with barium, or a gold thread). In yet other implementations, the radiopaque markers may be sewn into one or more pockets in the tubular graft portion 14, specially sized and positioned along the circumference to accept radiopaque material, where the pocket or pockets may then be sewn shut to retain the radiopaque material in the desired position on the stent graft 10.

Referring again to FIG. 1B, for a given row 18, the maximum number of radiopaque markers 16 for a given size stent graft 10 can be calculated by taking the circumference of the compressed (undeployed) stent graft multiplied by the percentage of the partial circumferential coverage for the row 18 divided by the diameter of a single marker 16. For example, assuming an 18 Fr (French circumference gauge) sheath (i.e., an 18.85 millimeter (mm) circumference) that will compress the stent graft down prior to deployment, and assuming the partial circumferential coverage of the markers for the stent graft 10 is 25% of the stent graft circumference (i.e., 90° of the total circumference), where each radiopaque marker is 1 mm in diameter, then the maximum number of markers for the row would be (18.84 mm*0.25)/1 mm=4.7 markers (4 markers after rounding down to get the largest whole number of markers).

Continuing with this hypothetical example (25% partial circumferential row length using 4 radiopaque markers for the row 20), the appropriate spacing between each marker 18 can be calculated by multiplying the circumference of the uncompressed stent graft 10 by the percentage of the partial circumferential coverage all divided by the number of markers minus one. If, for example, we assume the compressed stent graft described above expands to an 87.96 mm circumference when uncompressed (deployed), then the spacing between the markers in each row is (87.96 mm*0.25)/(4-1)=7.33 mm.

Although the example of FIGS. 1-2 shows 2 partial circumferential straight line rows of radiopaque markers that are axially offset and each occupy a unique partial circumferential portion of the circumference, other arrangements are contemplated. For example, the circumferential portion of each of the two exemplary rows may be at least 90° or may be less than 90° of the total stent graft circumference. Also, there may be more than two axially offset partial circumferential rows 18 of markers 16. The circumferential length may be more or less than 25% for each row 18. As one example, three axially offset and partially circumferential rows 18 of markers 16 may be used, where each row 18 has a unique 60° (16.67%) circumferential coverage.

Different combinations of partial circumferential lengths and different numbers of rows may be implemented. The length of the portion of the circumference occupied by one row of markers may be differ from row to row in some implementations. Also, the straight line arrangement of each row 18 illustrated may be replaced with a curved line or other angled arrangement of radiopaque markers in one or more of the rows. In yet other implementations, each row 18 may actually be a single continuous radiopaque marker that extends the partial circumferential length. Such a single radiopaque marker row may be in the form of a spiral coil sewn into the tubular graft material such that the spiral coil will compress and expand when the stent graft is expanded to a deployed position or contracted into an undeployed position. Alternatively, the single continuous marker may be only secured to the tubular graft portion 14 and/or the stent frame element 12 at the ends of the single continuous marker to allow that marker to bend and extend with expansion or contraction of the stent graft 10.

The arrangement of the circumferential offsets may vary. In one implementation, the rows 18 of markers 16 in their respective axially offset positions do not overlap circumferentially in terms of the portion of the 360° circumference of the stent graft 10 that they each occupy. The respective potions of the circumference each row occupies may be contiguous, where if merged into a single row rather than axially offset the markers would form an evenly spaced continuous line of markers, or they may be circumferentially discontinuous, where there would be an extra gap in the spacing of the markers between the end of the portion of the circumference occupied by a first row and the beginning of the portion of the circumference occupied by a second row. In yet other implementations, the portion of the circumference of one row 18 of markers 16 may partially overlap some of the same circumferential area of an axially offset second row of markers on the stent graft 10.

As has been described above, the use of two or more rows of radiopaque markers that are axially and circumferentially offset allows for unique x-ray projections of the stent graft device at rotational positions all around the 360° circumference of the stent graft. This may permit a user to place the stent graft in a desired rotational orientation before expanding the stent in the final location where it will be installed and thus may reduce guess work and inaccuracy. The closer spacing of the radiopaque markings in a given row while the stent graft is compressed may allow for relatively easy visualization via x-ray.

While various embodiments of the invention have been described, it will be apparent to those of ordinary skill in the art that many more embodiments and implementations are possible within the scope of the invention. Accordingly, the invention is not to be restricted except in light of the attached claims and their equivalents. Moreover, the advantages described herein are not necessarily the only advantages of the invention and it is not necessarily expected that every embodiment of the invention will achieve all of the advantages described.

The foregoing description of the inventions has been presented for purposes of illustration and description, and is not intended to be exhaustive or to limit the inventions to the precise forms disclosed. It will be apparent to those skilled in the art that the present inventions are susceptible of many variations and modifications coming within the scope of the following claims.

What is claimed is:

1. A stent graft device comprising:
a stent frame having a central axis;
a generally tubular graft member attached to the stent frame, wherein the stent graft device has a compressed state and an expanded state, wherein a diameter of the stent graft device in the expanded state is greater than that of the stent graft device in the compressed state;
a first row of radiopaque makers positioned along only a first portion of a circumference of the stent graft device, each of the radiopaque markers of the first row positioned on one of the stent frame or the graft member;
a second row of radiopaque markers positioned along only a second portion of the circumference of the stent graft device, each of the radiopaque markers of the second row positioned on one of the stent frame or the graft member, wherein the first row is axially and circumferentially offset from the second row; and
whereby a unique rotational position of the stent graft device is detectable via a pattern formed by the first and second rows of radiopaque markers in an image of the stent graft device.

2. The stent graft device of claim 1, wherein all radiopaque markers in the first row of radiopaque markers are attached to the graft member.

3. The stent graft device of claim 1, wherein all radiopaque markers in the first row of radiopaque markers are attached to the stent frame.

4. The stent graft device of claim 1, wherein all radiopaque markers in the first row of radiopaque markers and the second row of radiopaque markers are attached to the graft member.

5. The stent graft device of claim 1, wherein the first portion is circumferentially contiguous with the second portion of the circumference.

6. The stent graft device of claim 1, wherein the first portion is circumferentially discontinuous with the second portion of the circumference.

7. The stent graft device of claim 1, wherein a length of the first portion of the circumference is equal to a length of the second portion of the circumference.

8. The stent graft device of claim 7, wherein the length of the first portion of the circumference is at least 25 percent of a total circumference of the stent graft device.

9. A stent graft device comprising:
a plurality of stent frame elements arranged along a central axis;
a graft member attached with the stent frame elements, wherein the stent graft device has a compressed state and an expanded state, wherein a diameter of the stent graft device in the expanded state is greater than that of the stent graft device in the compressed state;
a plurality of rows of radiopaque markers positioned along a circumference of the stent graft device;
wherein each of the plurality of rows of radiopaque markers is axially spaced apart from each other of the plurality of rows with respect to the central axis, each of the radiopaque markers is positioned on one of the plurality of stent frame elements or the graft member and each of the plurality of rows has a circumferential length less than a total circumference of the stent graft device; and
whereby a unique rotational position of the stent graft device is detectable via a pattern formed by the plurality of rows of radiopaque markers in an image of the stent graft device.

10. The stent graft device of claim 9, wherein the plurality of rows are circumferentially offset from each other.

11. The stent graft device of claim 10, wherein none of the plurality of rows circumferentially overlaps any other of the plurality of rows.

12. The stent graft device of claim 11, wherein each of the plurality of rows extends a same distance along the circumference of the stent graft device.

13. The stent graft device of claim 11, wherein, in the compressed state, each radiopaque marker in a given one of the plurality of rows is spaced at a predetermined distance from an adjacent radiopaque marker in the given one of the plurality of rows.

14. A stent graft device comprising:
a radially expandable stent frame having a central axis;
a tubular graft member attached with a surface of the radially expandable stent frame, wherein the stent graft device has a compressed state and an expanded state, wherein a diameter of the stent graft device in the expanded state is greater than that of the stent graft device in the compressed state;
a plurality of rows of radiopaque material positioned on the stent graft device at axially offset locations, wherein each of the plurality of rows comprises at least one radiopaque marker positioned on the radially expandable stent frame or the tubular graft member and extending along a circumferentially unique portion of a total circumference of the stent graft device such that a fluoroscopic projection of the stent graft device on a two-dimensional display visually indicates a rotational orientation of the stent graft device in increments of 90 degrees or less.

15. The stent graft device of claim 14, wherein the plurality of rows comprises two rows.

16. The stent graft device of claim 15, wherein the circumferentially unique portion of the total circumference for each of the two rows comprises a respective 90 degree portion of the total circumference

17. The stent graft device of claim 14, wherein the at least one radiopaque marker comprises a plurality of radiopaque markers.

18. The stent graft device of claim 17, wherein the plurality of radiopaque markers are attached to the radially expandable stent frame.

19. The stent graft device of claim 17, wherein the plurality of radiopaque markers in each of the plurality of rows are arranged in a straight line along a respective different portion of the total circumference.

20. The stent graft device of claim 17, wherein a circumferential length of each of the plurality of rows of radiopaque markers is identical.
